(19) United States
(12) Patent Application Publication
(10) Pub. No.: US 2005/0131433 A1
(43) Pub. Date: Jun. 16, 2005

(54) Microkeratome cutting-blade assembly using staking and adhesive

(76) Inventor: Travis Michael Fisher, Kersey, PA (US)

Correspondence Address: Michael L. Smith;Bausch & Lomb Incorporated, One Bausch & Lomb Place, Rochester, NY, 14604-2701, US

(21) Appl. No.: 10/732,151

(22) Filed: Dec. 10, 2003

(51) Int. Cl. A61F 9/00

(52) U.S. Cl. 606/166

(57) ABSTRACT

A microkeratome cutting-blade assembly 31 includes a cutting-blade 34 attached to a blade holder 33. The cutting-blade 34 is attached to the blade holder 33 by heat staking and by adhesive.

### BACKGROUND OF THE INVENTION

1. Field of the Invention

The present invention relates to cutting-blade assemblies and specifically, cutting-blade assemblies for use in a microkeratome for use in ophthalmic surgery.

2. Description of the Related Art

Laser-Assisted In-situ Keratomileusis or LASIK surgery has become a widespread and effective eye correction surgical procedure in the last several years. Before a laser ablates a portion of a patient's corneal tissue to correct that patient's vision, a flap of the patient's cornea must be formed.

A typical cornea, on average, is about 520 microns thick. A typical flap thickness for the corneal flap, that is formed prior to laser ablation and LASIK surgery, is desired to be on the order of 160 to 200 microns. As is well known, these corneal flaps are made using microkeratomes that travel in a linear, arcuate, or even in a horizontally hinged path. A microkeratome typically cuts the corneal flap using a cutting-blade assembly made with standard razor blade stock available from any of numerous razor blade manufacturers, though other materials such as ceramics or plastics may be used. It is also typical that the cutting-blade is oscillated to aid in the cutting, while the cutting-blade is translated across the cornea to form a corneal flap.

A rather accurate measurement of the corneal thickness prior to LASIK surgery is obtainable through any number of known measurement methods, such as the use of an ORBSCAN™ Topography System available from Bausch & Lomb Incorporated. After the corneal thickness measurement has been obtained, depending on the surgeon's preference and the amount of correction needed, a flap thickness determination is then chosen by the surgeon.

Typically, in the prior art, each microkeratome comes with a variety of cutting heads, which are precisely manufactured to obtain different flap thicknesses, such as cuts of 160 microns, 180 microns, and 200 microns. Again, in the prior art, a single cutting-blade assembly has been used with these different precision cutting heads to obtain the different flap thicknesses.

One variation to this is from Med-Logics, Inc. Med-Logics currently manufactures LASIK blades, which consist of a piano or nominal length blade and a plus and a minus blade, wherein the blade extensions vary from the piano extension either plus or minus 20 microns. According to Med-Logics, this then allows the doctor to produce a flap of thinner or thicker thickness from the piano blade using a given cutting head.

A problem with all prior art microkeratome cutting-blade assemblies has been the consistency of the blade extension of the cutting head of the cutting-blade assembly. The blade extension is defined as the distance from the cutting tip of the blade to the nearest point of the blade holder. A microkeratome cutting head is precisely machined to applanate the cornea a given amount and to hold the blade holder within fairly tight tolerances. However to this point, the blade extension has not been held to a tight enough tolerance to give a consistent flap thickness cut. The criticality of the blade extension consistency has only recently become understood. The importance of blade extension consistency and a method of achieving such consistency are described in detail in co-pending U.S. patent application Ser. No. 10/334,358, filed Dec. 30, 2002, and entitled Microkeratome Cutting Blade Assembly and is hereby incorporated in its entirety by reference. It has always been a goal to provide a consistent and predictable flap thickness with a given cutting-blade in a given microkeratome cutting head.

The consistency of the flap thickness cut is crucial for several reasons. The reasons include that the laser ablation algorithm is based on the patient's need for correction and the amount of stromal bed left to be ablated after the flap has been created. This is critical to achieving an acceptable outcome for the patient. If too much corneal bed is ablated and not enough corneal bed thickness is left, the patient's intraocular pressure could cause serious change to the cornea. Conversely, if the corneal flap is too thin the flap could easily tear or it could be difficult to adequately correct the patient's vision without complications such as halos.

While it is easy to obtain a corneal thickness measurement before LASIK surgery, it has proven extremely difficult to measure corneal thickness of an eye with a corneal flap laid back over, and it is equally difficult to obtain a reliable corneal flap thickness measurement due to changes in hydration of the corneal flap and the cornea which occur quite rapidly under the surgical lights of an operating room.

If the corneal flap is thinner or thicker than desired by the surgeon and a patient's cornea is on the thin side to begin with, then serious complications could result from a flap that is thicker than desired. Therefore, it is desirable to provide a microkeratome cutting-blade assembly having a tightly controlled blade extension and to provide an easily accomplished method of producing such a tight blade extension.

It has been found that attaching a blade holder to a cutting-blade by known methods such as cold staking, heat staking, or adhesive bonding do not provide a robust enough bond to maintain the precise blade extensions desired under certain circumstances. Therefore, it would be desirable to provide an attachment between the blade holder and cutting-blade that is robust but yet economical to manufacture.

### BRIEF DESCRIPTION OF THE DRAWINGS

FIG. 1 is a side view of a prior art cutting-blade assembly;

FIG. 2 is a bottom view of FIG. 1;

FIG. 3 is a bottom view of a cutting-blade assembly in accordance with the present invention; and

FIG. 4 is a perspective view of an alternate embodiment of a cutting-blade assembly in accordance with the present invention;

### DETAILED DESCRIPTION OF THE DRAWINGS

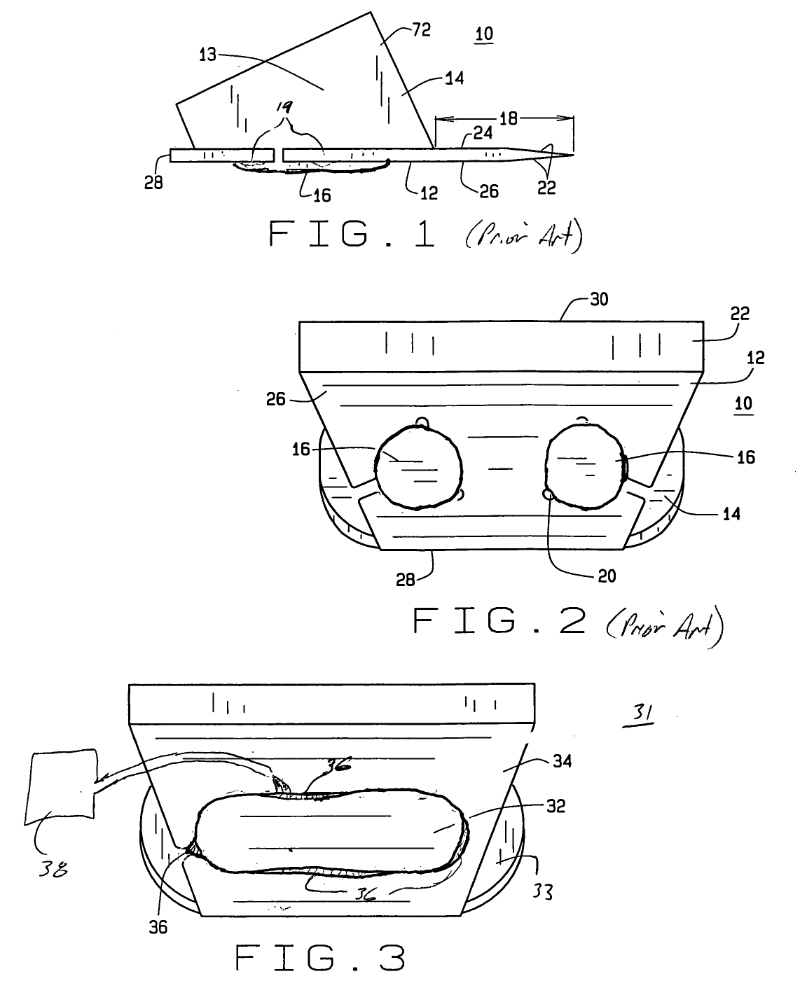

FIG. 1 shows a microkeratome cutting-blade assembly 10 in accordance with the present invention. Assembly 10 includes a cutting-blade 12 and a blade holder 14 attached to the cutting-blade 12. Preferably, blade holder 14 is attached to cutting-blade 12 through an aperture or through-hole in cutting-blade 12 (not shown) via post member 16 through a commonly known procedure such as heat staking. However, other means of attachment, such as cold staking, or other means are also possible. In addition, the aperture does not need to be a through-hole but rather could be mating indentations and raised portions in the blade holder and blade, as is known. Preferably, a blade extension represented by number 18 is controlled to within at least six (6) ten-thousandths of an inch of a target extension length for assisting and providing a consistent, predictable corneal-flap thickness. Blade extension 18 may also be measured from a front surface of holder 14 to a line parallel to the front surface and passing through the cutting tip of blade 12. Such tight tolerances and blade extensions may be very important as explained in detail in the above cited co-pending patent application. After staking, such as by the preferred heat staking, voids or gaps may form between the blade holder 14 and cutting-blade 12 as shown at 19. These gaps 19 are shown for illustrative purposes only. The gaps 19 in practice may not be seen from a visual inspection. These gaps reduce the strength of the possible bond between the holder 14 and blade 12. In fact, under certain conditions the bond may not be strong enough with just heat staking to hold the tight tolerances desired. These gaps are a by-product of achieving the desired tight blade extension tolerances. This is because the post member 16 must be moveable within the through-hole so that a precise blade extension can be achieved; there is simply not enough material in post 116 to fill all the gaps.

FIG. 2 is a bottom view of the assembly 10 of FIG. 1. The blade 12 is placed over post 16 of holder 14 as shown. The view of FIG. 2 is after the heat staking, and in this way notches 20 are partially seen. The purpose of notches 20 is to allow the material of post 16 upon heat staking to flow into the notches 20 and ensure attachment of the blade 12 to the blade holder 14 and the blade holder of the present invention. However, it may be preferable not to form notches 20 in blade 12. Preferably, blade holder 14 is made of Lubiloy™ and is molded or machined. Lubiloy™ is a polycarbonate material, which is preferred for blade holder 14, though any known suitable material is acceptable for blade holder 14, such as Delrin™. As previously discussed, cutting-blade 12 is preferably formed from razor blade stock widely available from a number of manufacturers, though a number of other materials are also possible.

FIG. 3 shows a cutting-blade assembly 31,in accordance with the present invention. A post member 32 of blade holder 33 is preferably heat or cold staked to cutting-blade 34 in a manner described in the above cited co-pending application to form cutting-blade assembly 31. Preferably, post member 32 is heat staked to cutting-blade 34 at between 350-425° F. at 10 psi and most preferably at 425° F. Adhesive 36 is then applied to blade assembly 31 to fill gaps between the blade holder 33 and cutting-blade 34 for forming a stronger bond than can be achieved with staking alone. Adhesive 36 is applied by any known method from a source 38 and is preferably #4304 available from Loctite but may be other adhesives suitable for surgical applications. Capillary action is believed to draw adhesive under the deformed post 32 and aids in adding lateral and axial strength to the assembly.

A gap must exist between the post 32 and through-holes (not shown) in cutting-blade 34 to allow the desired tight tolerance on blade extension to be achieved in a manufacturing environment. During assembly, as described in the cited co-pending application, the holder 33 moves relative to the blade 34 so that the desired blade extension can be achieved be staking the holder 33 to the blade 34. It has been found that because of the necessary gap between the post 32 and cutting-blade 34 heat staking will not sufficiently fill up the gap to create a strong enough bond. It has been found that the addition of adhesive can create a bond several times stronger than the bond achieved with staking or adhesive alone. In this way the tight blade extension tolerances desired may be maintained throughout operation and use of the cutting-blade assemblies.

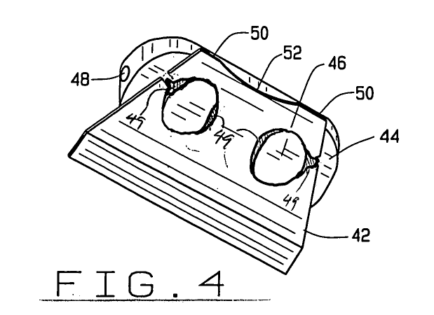

FIG. 4 shows an alternative embodiment of a microkeratome cutting-blade assembly in accordance with the present invention. A blade 42 is connected to a blade holder 44 via post 46, preferably by heat staking as described above, in addition to the use of adhesive 49. Adhesive 49 is preferably the same as adhesive 36. FIG. 4 also shows an insertion tool hole 48, such as known in the prior art and described in U.S. Pat. No. 6,051,009 to Hellenkamp, et al. Blade 42 has a back datum surface 50 and blade 42 is keyed by radius 52 being offset along back surface 50.

1. A microkeratome cutting-blade assembly comprises:
a blade holder;
a cutting-blade; and
wherein the cutting-blade is attached to the blade holder by heat staking and by adhesive.

2. A microkeratome cutting-blade assembly comprising:
a blade holder
a cutting-blade; and
wherein the cutting-blade is attached to the blade holder by cold staking and by adhesive.

3. A method of forming a microkeratome cutting-blade assembly comprising the steps of:
providing a blade holder;
providing a cutting-blade;
staking the cutting-blade to the blade holder to form a cutting-blade assembly;
applying adhesive to fill gaps between the blade holder and the cutting-blade for forming a stronger bond than can be achieved with staking alone.

4. The method of claim 3, wherein the staking step includes heat staking or cold staking.
